(19) United States
(12) Patent Application Publication
(10) Pub. No.: US 2019/0246986 A1
(43) Pub. Date: Aug. 15, 2019

(54) PORTABLE APPARATUS FOR IMAGING THE EYE

(71) Applicant: UNIVERSITY OF SOUTHERN CALIFORNIA, Los Angeles, CA (US)

(72) Inventors: Damien C. RODGER, Glendale, CA (US); Mark S. HUMAYUN, Glendale, CA (US); Peiyu WANG, Los Angeles, CA (US)

(73) Assignee: UNIVERSITY OF SOUTHERN CALIFORNIA, Los Angeles, CA (US)

(21) Appl. No.: 16/331,403

(22) PCT Filed: Sep. 8, 2017
(86) PCT No.: PCT/US2017/050726

§ 371 Date: Mar. 7, 2019

Related U.S. Application Data
(60) Provisional application No. 62/384,959, filed on Sep. 8, 2016.

(51) Int. Cl. A61B 5/00; A61B 3/117; A61B 3/15; A61B 3/12

(52) U.S. Cl. CPC A61B 5/6898; A61B 3/117; A61B 2560/0431; A61B 3/12; A61B 3/158

(57) ABSTRACT

A portable apparatus for imaging an eye in a patient includes a housing with a front portion and a rear portion, the front portion including an adapter sized to be placed over the eye of the patient. A lens is mounted within the housing, and a light source is provided within the housing to illuminate the eye. The rear portion of the housing is arranged to be mounted on a mobile device such that the lens is aligned with a camera associated with the mobile device for obtaining an image of the eye.

### CROSS-REFERENCE TO RELATED APPLICATIONS

This application claims the benefit of U.S. provisional application Ser. No. 62/384,959 filed Sep. 8, 2016, the disclosure of which is hereby incorporated in its entirety by reference herein.

### TECHNICAL FIELD

Embodiments relate to a portable apparatus for imaging the eye, such as for monitoring inflammation.

### BACKGROUND

Uveitis is defined as inflammation of the uvea, the middle layer of the eye (i.e., the pigmented vascular structures of the eye including the iris, ciliary body, and choroid), where symptoms include blurred vision, floaters, eye pain, redness and photophobia. Uveitis is reported to occur in 1 of every 1,000 people, at a rate of approximately 15-50 out of 100,000 people per year, accounting for 10% of cases of blindness in the United States (American Academy of Ophthalmology, 2014). In addition, it is most often a chronic and recurrent problem necessitating many return visits to eye care professionals for monitoring of disease, usually for life. It thus places a significant burden on society in terms of doctor visits, often several times a month, for the sole purpose of evaluating efficacy of treatment and titrating this treatment appropriately.

Anterior and panuveitis are the two most common types of uveitis, followed by posterior and intermediate uveitis. Exclusively anterior uveitis accounts for between 66-90% of uveitis cases. Both anterior and panuveitis, and to some extent intermediate and sometimes posterior uveitis, are characterized by, among other things, inflammation in the anterior chamber of the eye consisting of cells (typically floating individual white blood cells and pigmented cells) and flare, corresponding to a proteinaceous material, both of which are visible readily on slit lamp biomicroscopy in the clinic. White blood cells include PMN/neutrophil (12-15 μm diameter), lymphocyte (7-20 μm diameter), macrophage (21 μm diameter), and giant cells (40-50 μm diameter). Activity of the uveitis is most often determined by examining the anterior chamber for these two findings using the slit lamp, or sometimes using laser flare photometry.

Unfortunately, neither of these devices is portable, and both are very expensive. This poses significant limitations which prevent home measurement of disease activity for uveitis or for any other examination or monitoring of the eye. Current treatment paradigms consist of making a change in treatment regimen, sending the patient home, telling the patient to call in should there be significant worsening of the symptoms, and seeing them again anywhere from four to 12 weeks later to evaluate for changes. There are significant drawbacks to this approach. Often, patients do not feel symptomatically worse despite clear worsening in these two parameters upon follow up. Sometimes, patients fail to call even if they do notice changes, and then are seen at regularly scheduled follow up with much worse inflammation which has already caused scarring either in the front of the eye or the back of the eye, leading to vision loss through a variety of mechanisms. In other cases, the patient is unnecessarily seen earlier for a follow up appointment despite obvious improvement of these parameters.

### SUMMARY

In one embodiment, a portable apparatus for imaging an eye in a patient includes a housing with a front portion and a rear portion, the front portion including an adapter sized to be placed over the eye of the patient. A lens is mounted within the housing, and a light source is provided within the housing to illuminate the eye. The rear portion of the housing is arranged to be mounted on a mobile device such that the lens is aligned with a camera associated with the mobile device for obtaining an image of the eye.

In another embodiment, a portable apparatus for imaging an anterior chamber of an eye in a patient includes a housing with a front portion and a rear portion, the front portion including an adapter sized to be placed over the eye of the patient, the rear portion arranged to be mounted on a mobile device. A light source is provided within the housing to illuminate the eye, and a controller is provided in electrical communication with the light source. A lens is mounted within the housing, such that when the housing is mounted on the mobile device, the lens is aligned with a camera associated with the mobile device for obtaining an image of the eye. A software application is provided in communication with the mobile device for one or more of counting cells, detecting anterior chamber flare, or detecting vitreous haze in the image to detect uveitis.

In another embodiment, a portable apparatus for imaging an eye in a patient includes a housing with a front portion and a rear portion, the front portion including an adapter sized to be placed over the eye of the patient, the rear portion arranged to be mounted on a mobile device. A light source is provided within the housing to illuminate the eye. A lens is mounted within the housing, such that when the housing is mounted on the mobile device, the lens is aligned with a camera associated with the mobile device for obtaining an image of the eye. A software application is provided in communication with the mobile device for counting cells in the image, the software application sectioning out a portion of each image and averaging multiple images of equal concentration, using a morphological filtering process to detect individual cells in the image based on their shape, and using a secondary filtering process to detect individual cells in the image based on their size.

### BRIEF DESCRIPTION OF THE DRAWINGS

FIG. 1 is a photograph showing a perspective view of a portable imaging apparatus according to an embodiment;

FIG. 2 is a photograph showing a top perspective view of a lens support portion of the apparatus;

FIG. 3 is a photograph showing a side perspective view of the portable imaging apparatus;

FIG. 4 is a photograph showing a rear perspective view of the apparatus mounted on a mobile device;

FIG. 5 is a photograph showing a front perspective view of the apparatus mounted on a mobile device;

FIG. 6 is a photograph showing a rear perspective view of the apparatus mounted on a mobile device;

FIG. 7 is front perspective view of an apparatus housing according to an embodiment;

FIG. 8 is a rear perspective view of the apparatus housing of FIG. 7;

FIG. 9 is a rear perspective view of an apparatus housing according to another embodiment;

FIG. 10 is a front perspective view of the apparatus housing of FIG. 9;

FIG. 11 is a front perspective view of an apparatus housing according to yet another embodiment;

FIG. 12 is a rear perspective view of the apparatus housing of FIG. 11;

FIG. 13 is a front perspective view of an embodiment of the apparatus housing of FIG. 11 with the addition of a lens adapter and light source cover;

FIG. 14 is an end view of the embodiment of the apparatus housing of FIG. 13;

FIG. 15 is an illustration of the reflections that occur during a conventional slit lamp examination, and the space in between the reflections in which to look for particulate matter, cells, or “flare”;

FIG. 16A is a raw image of an eye taken with a mobile phone camera and slit beam illumination according to an embodiment;

FIG. 16B is a processed image showing 9 cells derived from the image area denoted by the square in FIG. 16A;

FIG. 17 is a photograph of a cell simulation using floating blue polyethylene microspheres obtained with the portable imaging apparatus;

FIG. 18 is a graph of cell simulation results using blue polyethylene microspheres such as shown in FIG. 17, where the actual manual particle count as a function of the density percentage is indicated with asterisks (*), and the software results are depicted for different threshold values;

FIG. 19 is a graph of the cell simulation results where a threshold value of 0.3 is plotted against the manual particle count; and

FIG. 20 is an image of the optic disc obtained by the system disclosed herein.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Embodiments disclosed herein include a portable apparatus for imaging the eye, such as for monitoring eye inflammation (e.g., uveitis). As explained above, the severity of uveitis is clinically determined by the number of floating white blood cells in the anterior chamber, or by determining the amount of anterior chamber flare or vitreous haze seen by slit lamp examination. The apparatus applies similar principles as slit lamp cell grading diagnostic techniques (Standardization of Uveitis Nomenclature (SUN) Uveitis Working Group criteria; Jabs et al., Am J Ophthalmol 2005; 140(3): 509-16), but is portable so that the patient can take the apparatus home, self-monitor their eye condition, and communicate with their physician as necessary.

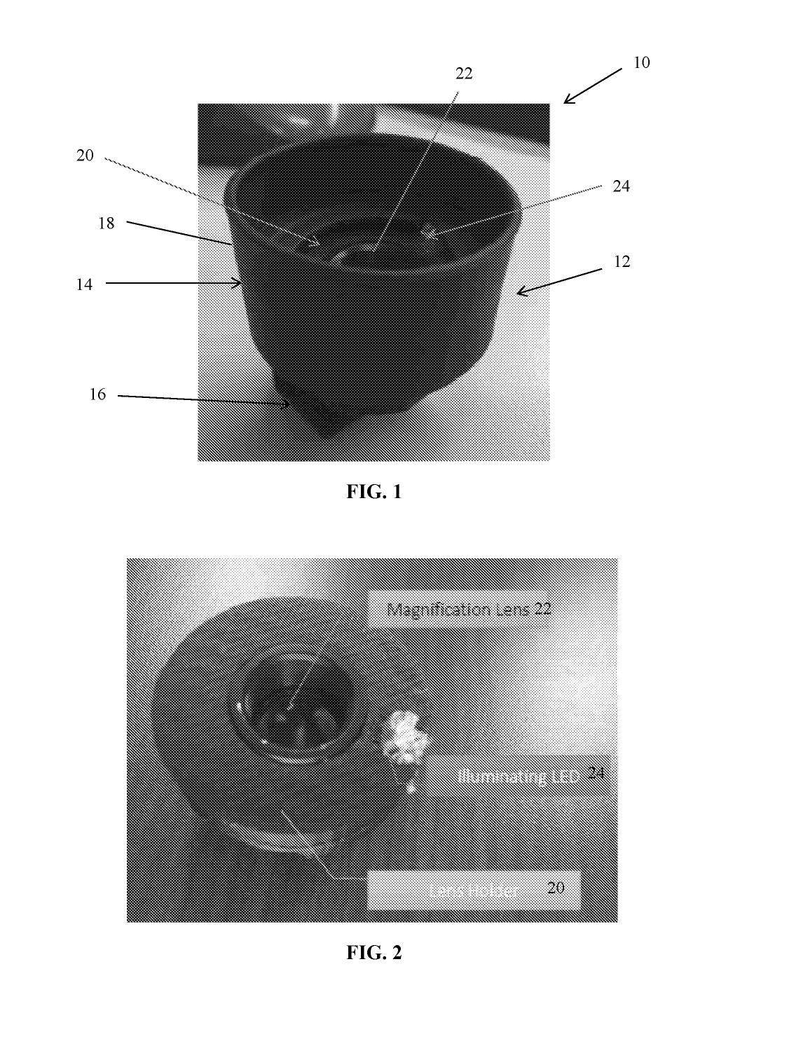

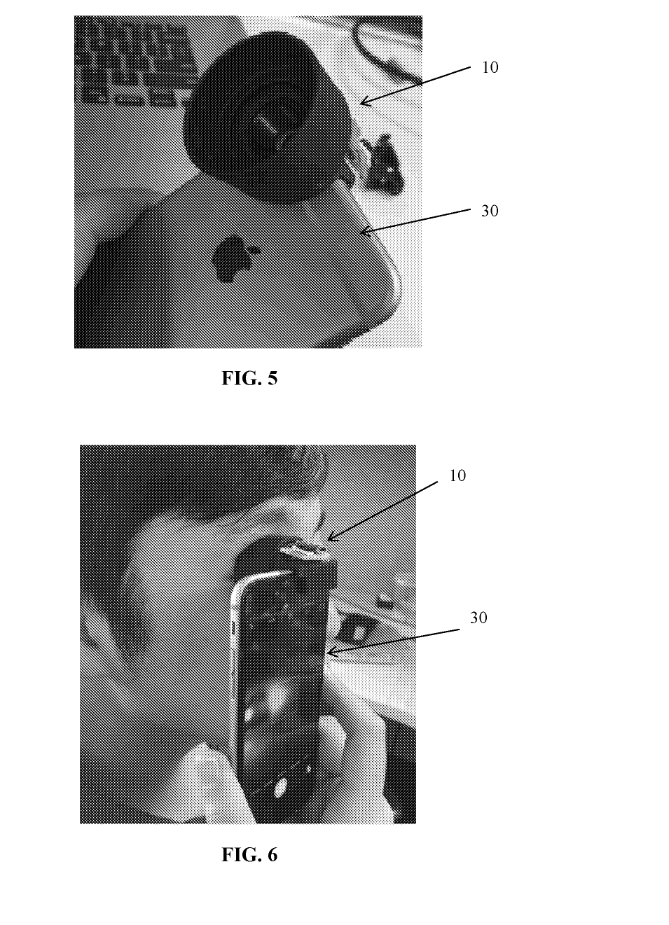

With reference to FIGS. 1-6, an embodiment of a portable monitoring apparatus is illustrated and designated generally by reference numeral 10. The apparatus 10 includes a housing 12 with a front, imaging portion 14 and a rear, mounting portion 16. The front portion 14 includes an adapter, such as a hollow eye cup 18, sized to be placed over a patient's eye area in order to block ambient light. While a generally cylindrical configuration of the eye cup 18 is shown herein, it is understood that the eye cup 18 is not limited to this shape.

As best shown in FIGS. 1 and 2, a lens support 20 is disposed within the front portion 14 of the housing 12, set inward from the eye cup 18. The lens support 20 houses a magnification lens 22 for imaging the eye. In one embodiment, the lens 22 may be a bi-convex lens with 0.5 in. (12.7 mm) diameter, and a 15 mm focal length (e.g. Thorlabs LB1092-A-N-BK7). In another example, a lens with a diameter between 6 mm-9 mm and a focal length between about 15-20 mm may be used. The 15 mm focal length may be utilized for evaluating inflammation in the anterior chamber of the eye. For different areas of the eye, such as the vitreous cavity, a lens with a different focal length may be substituted.

A light source 24 may be mounted on the lens support 20 or on the housing 12 to provide appropriate illumination to the eye for imaging. In one embodiment, the light source 24 is a narrow spatial distributed blue LED. In another embodiment, the light source 24 may be a white LED, which may be round with a domed top (e.g. 5.00 mm) with a viewing angle of 15 degrees. The light source 24 is useful in detecting white blood cells, while light sources with different wavelengths can be used for detecting other types of cells, such as pigmented cells. It is possible that two different light sources 24 could be used, one for illumination (e.g. white LED), and one as a fixation point for the patient (e.g., blue LED).

In contrast with conventional slit lamp illumination, where the beam standard is 1 mm×1 mm at 45 degrees, the light source 24 disclosed herein can utilize a beam of different size and angle of illumination. For example, a beam larger than 1 mm×1 mm could be used, as well as a different incident light angle such as, but not limited to, 30-65 degrees, as such a configuration may facilitate obtaining more data. In one non-limiting embodiment, a fixed 53 degree angle may be selected.

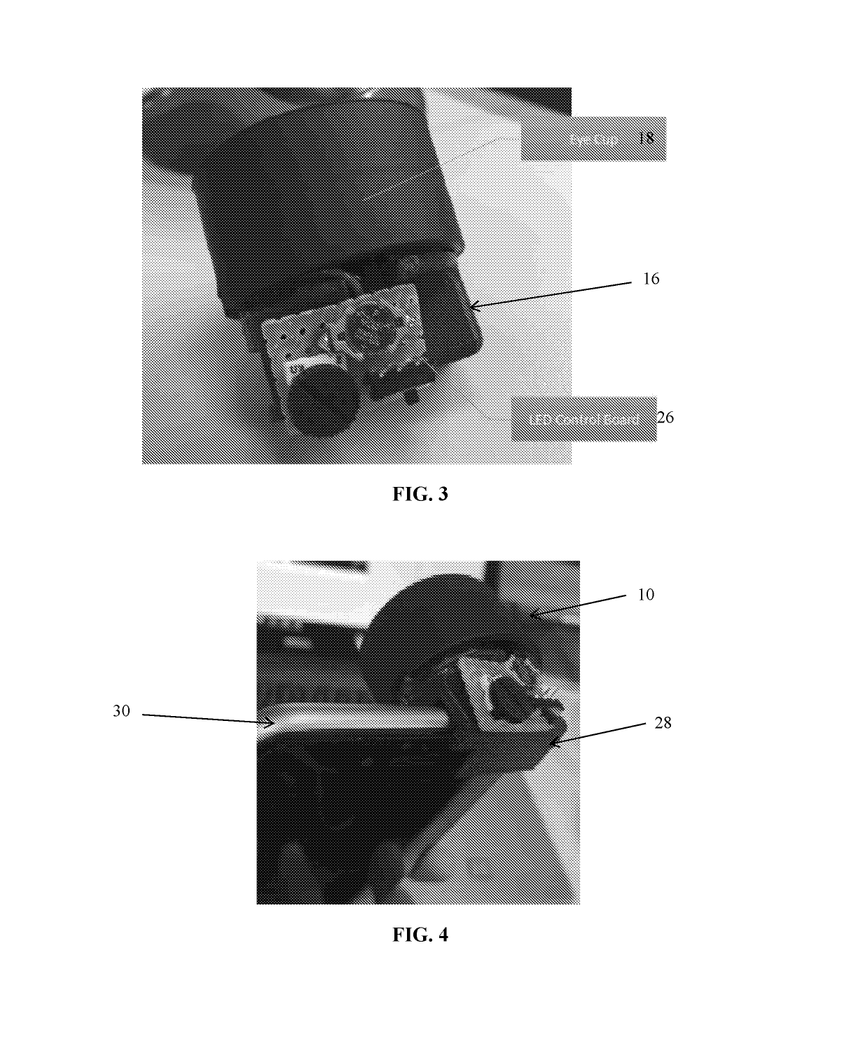

As shown in FIGS. 3 and 4, a controller 26 can be provided in electrical communication with the light source 24 for controlling the brightness of the light source 24. The controller 26 may be used, for example, to vary the brightness of the light source 24 in order to best image the cornea given a particular condition or disease state. The controller 26 can be mounted to the rear portion 16 or in any other suitable location on or within the housing 12. The controller 26 can receive input from a software application, which is described further below.

With reference to FIGS. 4-6, the rear portion 16 may include a mounting flange 28 for mounting to a mobile device 30 having a camera, such as a mobile phone. The apparatus 10 may be positioned so that the lens 22 is aligned with the camera for imaging. It is contemplated that the rear portion 16 may also include a clamping mechanism to secure the apparatus 10 in position with respect to the mobile device 30.

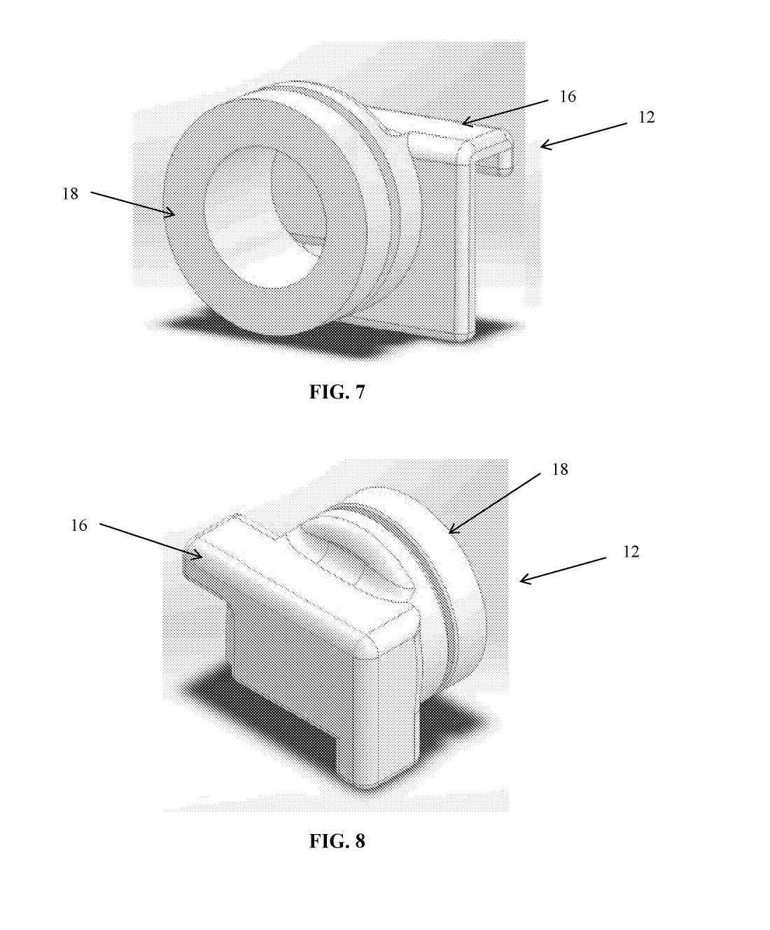

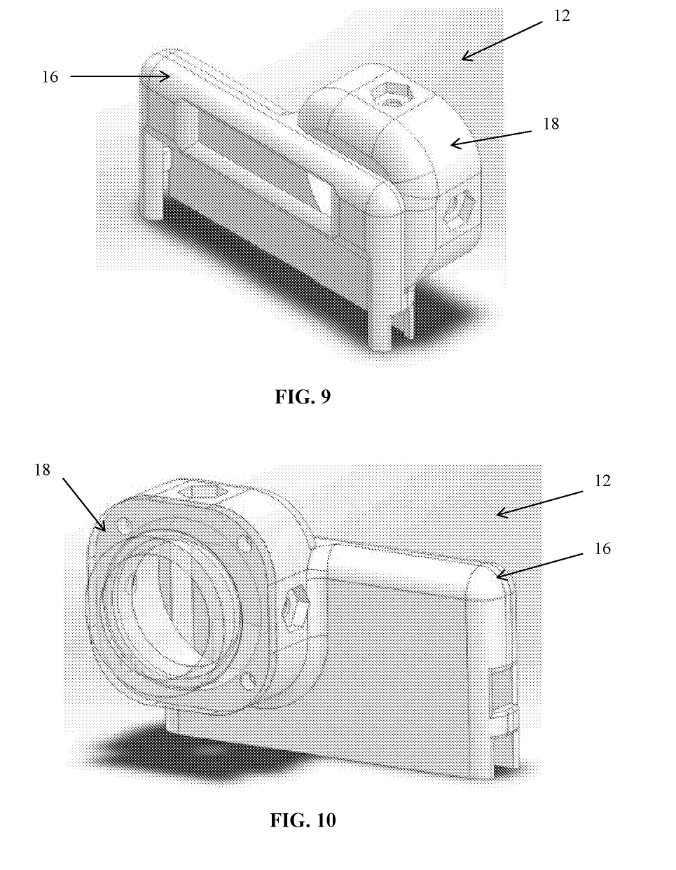

While a particular configuration of the apparatus 10 has been described above with respect to FIGS. 1-6, this configuration is not intended to be limiting. For example, FIGS. 7-8 illustrate another embodiment of the housing 12, while FIGS. 9-10 illustrate yet another possible embodiment of the housing 12.

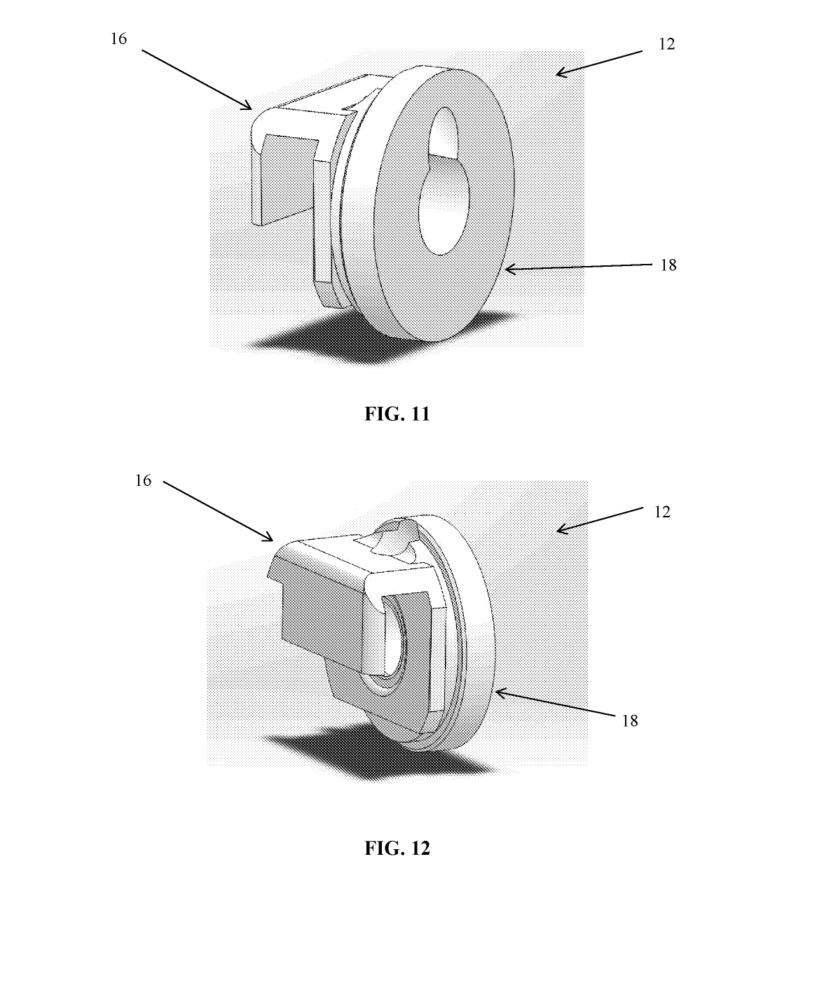

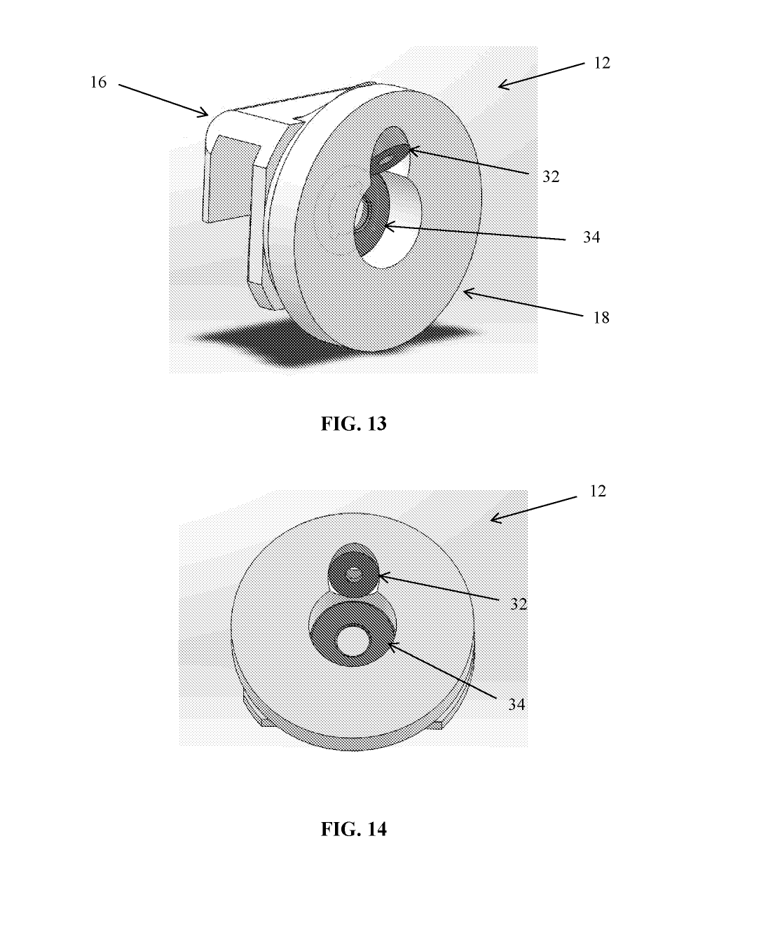

With reference to FIGS. 11-14, another embodiment of the housing 12 is depicted which may accommodate a light source cover 32 and a lens adapter 34. The light source cover 32 may be mounted to extend forward of the light source 24 within the adapter 18 or lens holder 20 portions of the housing 12, and may be used to control the shape and size of the light beam. For example, the beam from the light source 24 could be shaped into a circular, square or a slit beam. It is contemplated that the light source cover 32 could be adjustable such that the characteristics of the illumination could be modified during the imaging procedure. The lens adapter 34 (e.g. Thorlabs LMRA06 or LMRA09) may be mounted within the housing 12, such as in the lens holder 20, and may allow the position of the lens 22 to be adjustable to allow for control of the lens prior to or while conducting the examination.

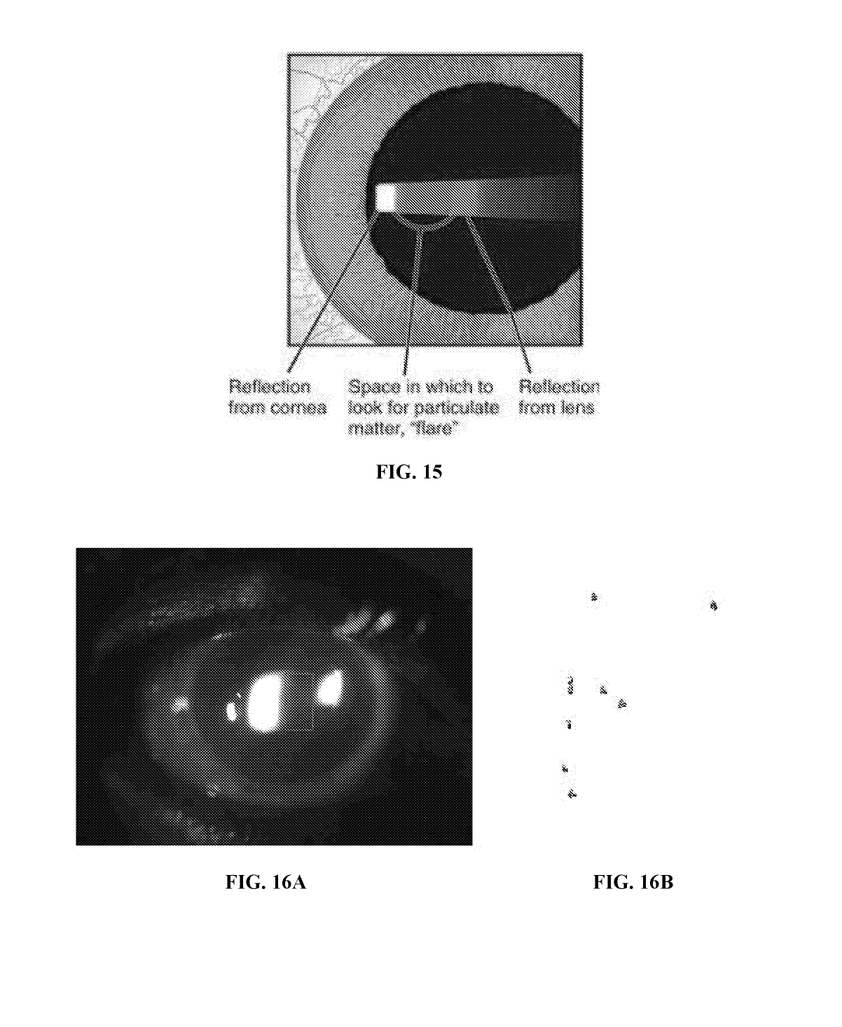

With reference to FIG. 15, during a slit lamp examination, there is a reflection from the cornea and a reflection from the lens, and between those reflections is a space in which to look for particulate matter, cells, or “flare”. FIG. 16A is a raw image of an eye taken with a mobile phone camera and slit beam illumination according to embodiments disclosed herein, and FIG. 16B is a processed image showing 9 cells derived from the image area denoted by the square in FIG. 16A. In this example, the patient had 1+ cell on exam (defined as 5-15 per high power field) according to SUN criteria, showing good correlation of processed data and exam findings.

A software application is provided in communication with the mobile device 30 for sectioning the eye image and counting cells in a repeatable manner. The cell count data can be compared to prior results and used to direct treatment. In one embodiment, particle counts are done by sectioning out the 1/9 middle part of each image and then averaging multiple images under the same concentration. Anisotropic diffusion may be utilized to achieve sectioning of the cornea. Image recognition algorithms can also be used to determine the amount of anterior chamber flare or vitreous haze for identifying uveitis.

Prior to performing final particle counts, additional image processing procedures may be performed. One such procedure may include converting the image to grayscale from its original RGB format of red, green and blue layers. In one embodiment, only one of the RGB layers may be used, such as for filtering out pigment cells that cause miscalculation based on their color.

A top hat filter is a morphological filter which filters out structures based on their shape. In one embodiment, a morphological filtering process, such as using a top hat filter, may be used in which most of the background of the image is removed, and only the “dots” in the image are preserved. This could be a working filtering process for segmenting valid information before cell counts. After top hat filtering, unwanted cell structures may still exist. Therefore, a secondary filtering process may be performed based on the size of the particles, where articles that have unrealistic widths or sizes will be ignored.

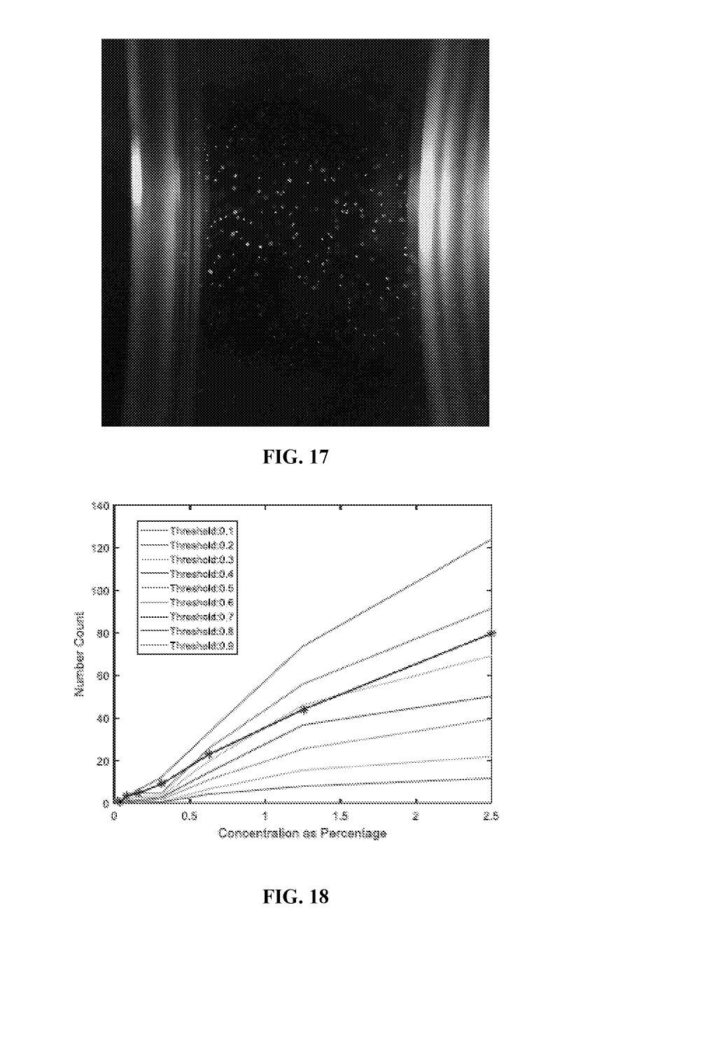

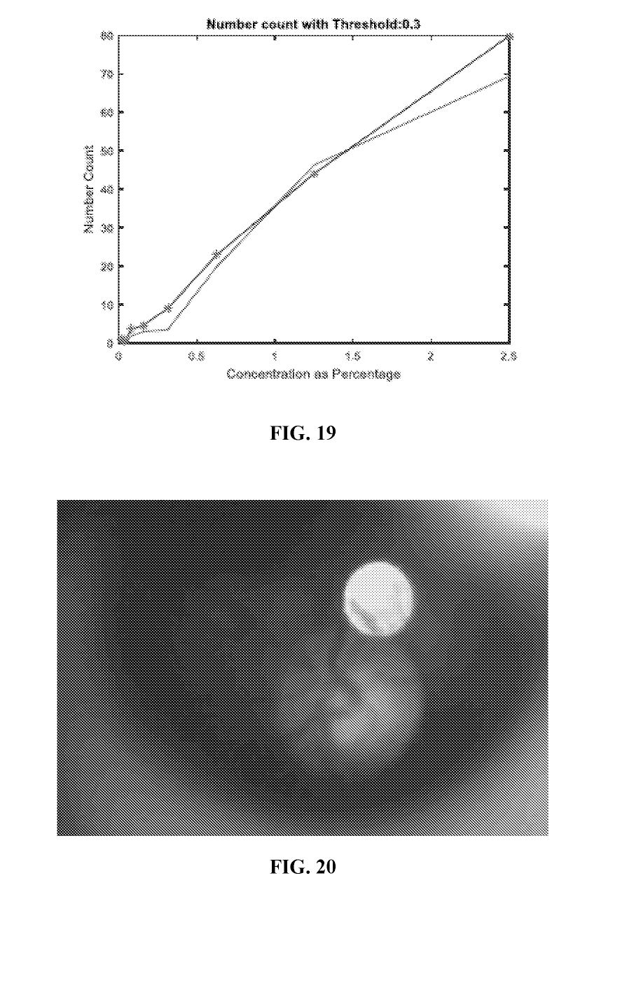

FIG. 17 is a photograph of a cell simulation using floating blue polyethylene microspheres of 10-27 μm diameter obtained with the portable imaging apparatus 10. FIGS. 18 and 19 depict the results of preliminary studies conducted with a cell simulation comprising blue polyethylene microspheres as in FIG. 17, where the center 1/9 portion of the image was used and the disclosed algorithms/filters for counting the beads were compared to manual counting. In the graph of FIG. 18, the actual manual particle count as a function of the density percentage is indicated with asterisks (*), and then the software results are depicted for different threshold values (different noise vs. signal levels). In the graph of FIG. 19, a threshold value of 0.3 is plotted against the manual particle count, demonstrating the good correlation between actual and software-calculated values.

The software application can be further used to optimize photography parameters, transfer the images and data to the physician, assist in diagnosing conditions, and provide instructions to the patient. Depending on the results of the images, a physician can communicate instructions to the patient to make a new appointment, to delay an appointment due to an improved condition, to accelerate the time frame for an appointment due to a worsened condition, or any other instructions related to the patient's care.

The potential of a market for the portable imaging apparatus disclosed herein is enormous, for example due to the need for a portable yet accurate way to monitor eye inflammation such as uveitis using nomenclature that all ophthalmologists understand. In addition to the uveitis population, local ophthalmology clinics and uveitis specialists, the apparatus could be used to monitor the eye condition of postoperative patients, such as following cataract surgery.

The retina is an essential part of the human eye and can be affected by many pathologic changes, and the apparatus 10 disclosed herein can also be used for imaging the retina. A virtual image is created by a lens 22 accompanied by the natural structures of the eye (the crystalline lens and the cornea), and is captured by the camera of the mobile device 30. An example image is shown in FIG. 20.

For imaging the retina, one illumination option is side illumination, where an illumination source is placed beside the camera within a short distance. Another illumination option is direct vertical illumination, where a light source with a very small dimension is placed in front of the camera. If the light source is small enough and very close to the camera, the view will not be blocked. Still another illumination option is trans-sclera illumination where instead of illuminating through the iris, illumination could be achieved through the sclera. This type of illumination would avoid noise and reflective glare.

In one embodiment, the lens 22 may be a bi-concave lens with a diameter of 1.0 in. (25.4 mm) and a focal length of −50 mm. In this manner, a virtual image is created as in a direct ophthalmoscope. However, the lens 22 is not limited to this diameter and focal length, and may have the same parameters as the lens used for anterior chamber examination. It is also contemplated that the housing 12 may interchangeably accommodate different lenses 22 and include different light sources 24 required for imaging both the retina and the anterior chamber, as well as possibly other eye structures.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A portable apparatus for imaging an eye in a patient, comprising:
a housing with a front portion and a rear portion, the front portion including an adapter sized to be placed over the eye of the patient;
a lens mounted within the housing; and
a light source provided within the housing to illuminate the eye;
wherein the rear portion is arranged to be mounted on a mobile device such that the lens is aligned with a camera associated with the mobile device for obtaining an image of the eye.

2. The apparatus of claim 1, wherein the light source includes a white or blue LED.

3. The apparatus of claim 1, wherein the adapter is generally cylindrical.

4. The apparatus of claim 1, further comprising a controller in electrical communication with the light source.

5. The apparatus of claim 4, wherein the controller is mounted to the rear portion of the housing.

6. The apparatus of claim 1, wherein the rear portion includes a mounting flange for mounting to the mobile device.

7. The apparatus of claim 1, further comprising a light source cover which cooperates with the light source to control the shape and size of the light beam.

8. The apparatus of claim 1, further comprising a lens adapter which cooperates with the lens to allow the position of the lens to be adjustable.

9. The apparatus of claim 1, further comprising a software application in communication with the mobile device for one or more of counting cells, detecting anterior chamber flare, or detecting vitreous haze in the image.

10. The apparatus of claim 9, wherein counting cells includes sectioning out a portion of each image and averaging multiple images of equal concentration.

11. The apparatus of claim 9, wherein counting cells includes using a morphological filtering process to detect individual cells in the image based on their shape.

12. The apparatus of claim 9, wherein counting cells includes a filtering process to detect individual cells in the image based on their size.

13. A portable apparatus for imaging an anterior chamber of an eye in a patient, comprising:
a housing with a front portion and a rear portion, the front portion including an adapter sized to be placed over the eye of the patient, the rear portion arranged to be mounted on a mobile device;
a light source provided within the housing to illuminate the eye;
a controller in electrical communication with the light source;
a lens mounted within the housing, such that when the housing is mounted on the mobile device, the lens is aligned with a camera associated with the mobile device for obtaining an image of the eye; and
a software application in communication with the mobile device for one or more of counting cells, detecting anterior chamber flare, or detecting vitreous haze in the image to detect uveitis.

14. The apparatus of claim 13, wherein the rear portion includes a mounting flange for mounting to the mobile device.

15. The apparatus of claim 13, further comprising a light source cover which cooperates with the light source to control the shape and size of the light beam.

16. The apparatus of claim 13, further comprising a lens adapter which cooperates with the lens to allow the position of the lens to be adjustable.

17. The apparatus of claim 13, wherein counting cells includes sectioning out a portion of each image and averaging multiple images of equal concentration.

18. The apparatus of claim 13, wherein counting cells includes using a morphological filtering process to detect individual cells in the image based on their shape.

19. The apparatus of claim 13, wherein counting cells includes a filtering process to detect individual cells in the image based on their size.

20. A portable apparatus for imaging an eye in a patient, comprising:
a housing with a front portion and a rear portion, the front portion including an adapter sized to be placed over the eye of the patient, the rear portion arranged to be mounted on a mobile device;
a light source provided within the housing to illuminate the eye;
a lens mounted within the housing, such that when the housing is mounted on the mobile device, the lens is aligned with a camera associated with the mobile device for obtaining an image of the eye; and
a software application in communication with the mobile device for counting cells in the image, the software application sectioning out a portion of each image and averaging multiple images of equal concentration, using a morphological filtering process to detect individual cells in the image based on their shape, and using a secondary filtering process to detect individual cells in the image based on their size.
